(19) United States
(12) Patent Application Publication
(10) Pub. No.: US 2014/0364727 A1
(43) Pub. Date: Dec. 11, 2014

(54) METHOD OF THERMO-ACOUSTIC TOMOGRAPHY AND HYPERTHERMIA

(71) Applicant: THE BOARD OF REGENTS FOR OKLAHOMA STATE UNIVERSITY, STILLWATER, OK (US)

(72) Inventor: Daqing Piao, Stillwater, OK (US)

(73) Assignee: The Board of Regents for Oklahoma State University, Stillwater, OK (US)

(21) Appl. No.: 14/375,265

(22) PCT Filed: Jan. 30, 2013
(86) PCT No.: PCT/US2013/023821

§ 371 Date: Jul. 29, 2014

Related U.S. Application Data
(60) Provisional application No. 61/592,324, filed on Jan. 30, 2012.

(51) Int. Cl. A61B 5/00; A61B 5/05

(52) U.S. Cl. CPC A61B 5/0095; A61B 5/0522
USPC 600/431

(57) ABSTRACT

A method includes providing a pulsed magnetic field, exposing a tissue mass to the pulsed magnetic field, and receiving an ultrasonic signal from a region of the tissue imbued with magnetic particles.

### CROSS REFERENCE TO RELATED APPLICATION

This application claims the benefit of U.S. Provisional Application No. 61/592,324 filed Jan. 30, 2012, herein incorporated by reference in its entirety for all purposes.

### STATEMENT REGARDING FEDERALLY SPONSORED RESEARCH OR DEVELOPMENT

This invention was made with U.S. Government support under NIH Grant Number CA136642 awarded by the National Institutes of Health. The Government has certain rights in the invention.

### FIELD OF THE INVENTION

This disclosure relates to tomographic imaging in general and, more specifically, to magnetic tomographic imaging.

### BACKGROUND

When tissues are illuminated with various kinds of radiation, the radiative energy may be converted to heat within the tissues (living or otherwise). Such heating can be used therapeutically on its own or along with drugs or treatments that are activated or augmented by heating.

Heated tissue may also expand relative to the surrounding tissues when heated. If the illumination is applied in a periodic fashion, the illuminated tissues can expand and contract with the application of the illumination. Depending upon the period of the illumination an ultrasonic signal can be generated from the illuminated tissues. Previously, various forms of electromagnetic radiation (including visible light) have been used for the illumination. Of course, the depth or range of the illumination in such cases is limited due to the high level of attenuation of light when travelling through most tissues. Microwave illumination has also been used, and has increased penetration depending upon the frequency, but illumination along the depth of imaging has been non-uniform.

What is needed is a system and method for addressing the above, and related, issues.

### SUMMARY OF THE INVENTION

The invention of the present disclosure, in one aspect thereof, comprises a method including providing a pulsed magnetic field, exposing a tissue mass to the pulsed magnetic field, and receiving an ultrasonic signal from a region of the tissue imbued with magnetic particles. The magnetic particles may comprise super-paramagnetic iron oxide nanoparticles.

In some embodiments, the pulsed magnetic field is pulsed by being activated for a recurring period and deactivated for a second recurring period, the activated period comprising an amplitude modulated magnetic field. The amplitude modulated magnetic field may have a frequency of about 10 MHz. The activated period may be about one microsecond in duration. Similarly, the deactivated period may be about one microsecond in duration. The activated period may have a duration including at least one complete cycle of the alternating magnetic field.

In some embodiments, the pulsed magnetic field is pulsed by being activated for a recurring period and deactivated for a second recurring period, the activated period comprising a frequency modulated magnetic field. The frequency modulated magnetic field may include a frequency that varies up to a high frequency of about 10 MHz.

The invention of the present disclosure, in another aspect thereof, comprises a method that includes attaching magnetic particles to a target tissue region within a tissue mass, exposing the tissue mass to a field pulse enveloped alternating magnetic field, and reading an ultrasonic signal generated by the target tissue region containing the magnetic particles.

In some embodiments, attaching magnetic particles further comprises attaching magnetic nanoparticles. The magnetic nanoparticles may comprise super-paramagnetic iron oxide nanoparticles. The method may include generating a map of the target tissues based on the ultrasonic signal generated by the magnetic particles. The pulse alternating magnetic field may comprise an amplitude modulated portion, or a frequency modulated portion. A period when the magnetic field is active may have a duration of at least one cycle of the alternating magnetic field.

The invention of the present disclosure, in another aspect thereof, comprises a magnetic field generator configured to provide a pulse enveloped alternating magnetic field to a tissue mass having a target region containing magnetic particles, the pulse enveloped alternating magnetic field, and an ultrasonic transducer that receives an ultrasonic signal from the tissue mass representative of the target region resulting from heating and cooling of the target region from the pulse enveloped alternating magnetic field. In some embodiments, the magnetic field generator provides a pulse enveloped alternating magnetic field having an amplitude modulated field. In other embodiments, the magnetic field generator provides alternating magnetic field having a frequency modulated field.

### BRIEF DESCRIPTION OF THE DRAWINGS

FIG. 1 is a schematic diagram illustrating cyclic expansion and contraction of tissue under pulsed illumination.

FIG. 2 is schematic diagram illustrating pulsed heating and expansion of tissue by pulsed illumination.

FIG. 3 is a schematic diagram and temperature chart illustrating the effect of exposure to an alternating-magnetic-field on magnetic particles.

FIG. 4 is a schematic diagram illustrating the effect of exposure to a pulsed alternating magnetic field on magnetic particles.

FIG. 5(A) is a graph of heat dissipation of magnetic nanoparticles over time when excited by a continuous alternating magnetic field.

FIG. 5(B) is a graph of heat dissipation of magnetic nanoparticles over time when excited by an amplitude modulated alternating magnetic field.

FIG. 5(C) is a graph of heat dissipation of magnetic nanoparticles over time when excited by a frequency modulated alternating magnetic field.

FIG. 6 is a schematic diagram of a device constructed to provide electromagnetic fields to test subjects containing magnetic nanoparticles.

FIG. 7(A) is a graph of the temperature rise magnetic nanoparticles under magnetic fields of various frequencies.

FIG. 7(B) is an extrapolation of the data of FIG. 7(A).

FIG. 8(A) is a graph of volumetric heat dissipation versus depth.

FIG. 8(B) is a graph of heat dissipation of magnetic nanoparticles over time.

### DETAILED DESCRIPTION OF THE PREFERRED EMBODIMENTS

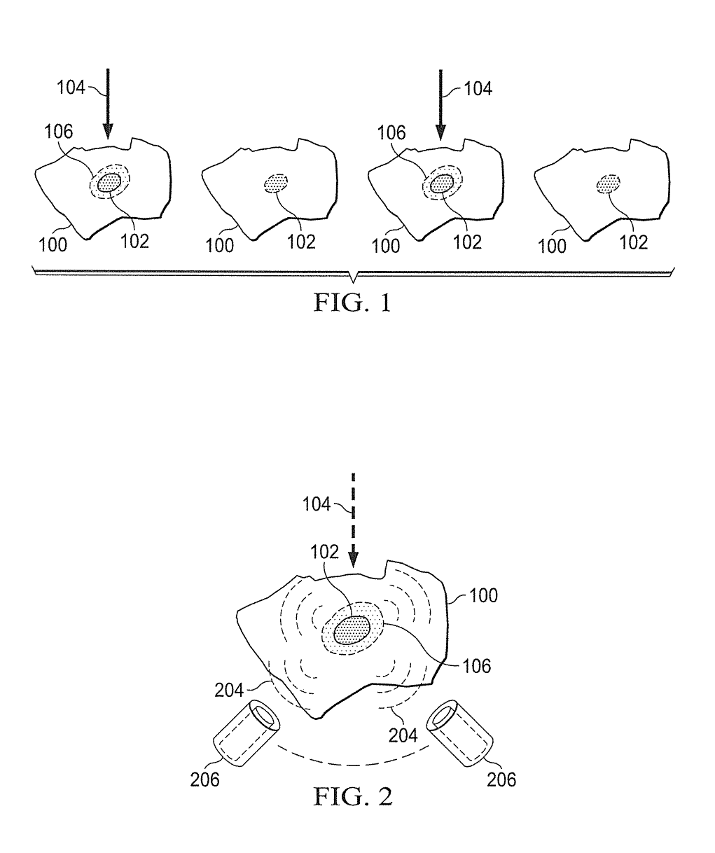

The thermo-acoustic effect, as regarding living tissue, refers to the generation of an acoustic signal due to elastic expansion of the tissue as the tissue is heated by pulsed illumination of certain types of radiation. Referring now to FIG. 1, a tissue mass 102 may have a portion 102 that is heated and expanded by such radiation 104 and attain a larger expanded size 106 within the mass 102. When the illumination stops, the mass 102 returns to its original size. Endogenous or exogenous tissue components can absorb radiation which is converted to heat. If the radiation is turned on and off repetitively, the tissue will expand and contract at a cycle following the ON/OFF cycle of the radiation as shown (from left to right).

Referring now to FIG. 2, if the ON-duration of the radiation is sufficiently short (e.g., at a micro-second level), it may be considered a pulsed illumination 202. The rapid expansion of the tissue 102 and the following contraction give rise to acoustic signal 204 in a range that can be detected by an ultrasonic transducer 206. These acoustic signals can then be used to map the distribution of the heat-generating region of the tissue 102. Mapping tissues using acoustic waves is the basis of the ultrasound devices used in hospitals.

Generation of thermo-acoustic signals from tissue requires the following conditions to be met: (1) the energy of the localized radiation can be converted to heat by absorption; and (2) the localized radiation changes rapidly in time. Continuous radiation at a fixed energy deposition rate causes steady temperature rise, which does not give rise to the acoustic signal. Only rapid rise/fall of the temperature could generate the acoustic signal.

One difference in the heat-generating illumination between photo-acoustic tomography or opto-acoustic tomography and microwave-induced thermo-acoustic tomography leads to an important difference in the contrast mechanism between these two techniques. Hemoglobin and melanin contribute to the main optical absorption in photo or opto-acoustic tomography, while ion and water concentration is responsible for microwave-induced thermo-acoustic contrast.

Recently there has been a significant interest in applying PAT (OAT) and MI-TAT techniques in biomedical imaging application, such as breast cancer imaging, brain structural and functional imaging, foreign body detection, deep tumor imaging, and for molecular imaging.

One advantage of both PAT (OAT) and MI-TAT is that specific imaging contrast invisible to ultrasound is acquired at ultrasonic resolution. Because tissue scattering of ultrasound is weak, and ultrasound has a speed of approximately 1.5 mm/μs in tissue and penetrates centimeters in tissue, a MHz range ultrasound detection results in a millimeter-level image resolution over centimeters of tissue. Thus, the limit of imaging depth is usually set by the limit of illumination depth.

One disadvantage of PAT is that it uses light to illuminate/excite the subject. As tissue-scattering of light is very strong, light is attenuated exponentially along the depth and becomes diffusive. Therefore light illumination along the depth of imaging (usually several centimeters) is significantly non-uniform. PAT is also limited in imaging through blood-rich organs such as a heart or a liver because the light is strongly attenuated by hemoglobin.

One disadvantage of MI-TAT results from its use of microwave illumination. Tissue-attenuation of microwave is a function of microwave frequency the higher the frequency is, the less tissue penetration. For the ˜3 GHz microwave typically used in MI-TAT, the imaging depth is several centimeters. Furthermore, the illumination along the depth of imaging is significantly non-uniform. TAT tissue contrast is interpreted as coming from the varying water content of the tissues; however the clinical relevance of this contrast mechanism needs to be further evaluated.

Under an alternating-magnetic-field (AMF) in the frequency range of 10 s of KHz to a few MHz, micron-scale or nanometer-size magnetic particles undergo relaxation processes, including hysteresis, Brownian relaxation, and Neel relaxation. As a result, the temperature of magnetic particle increases, often in a dramatic rate. As certain magnetic particles, such as super-paramagnetic iron oxide (SPIO), can be conjugated to disease-specific ligands, the magnetic particles can be targeted to a diseased site. Applying AMF will then increase the temperature of tissue at the location of the particles. Such mechanism has been used in localized hyperthermia for cancer treatment, controlled drug-release, etc.

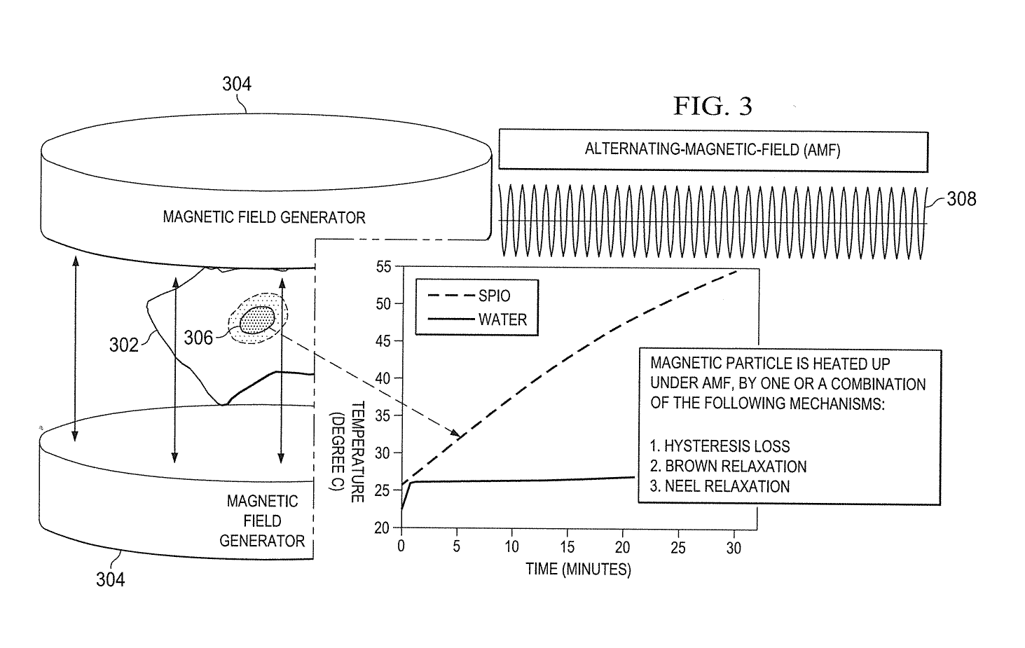

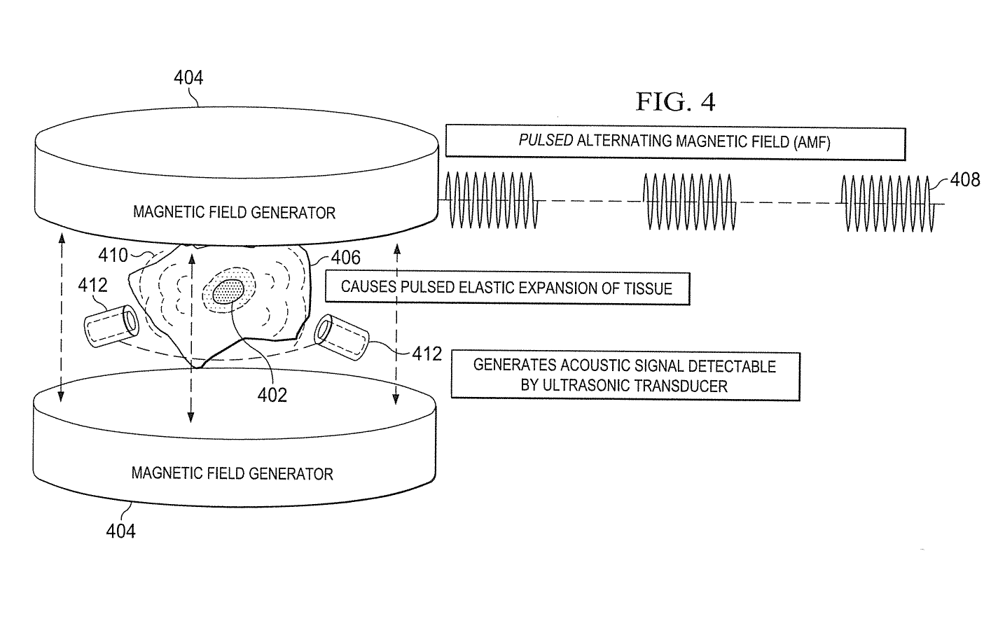

FIG. 3 illustrates the operation of the method of utilizing an AMF to create a temperature increase in tissue. A tissue mass 302 is exposed to a magnetic field generator 304. The tissue mass contains a portion 306 containing a concentration of magnetic particles. The AMF 308 (inset) creates an increase in temperature of the portion 306 of the mass 302 containing magnetic particles as illustrated in the lower inset graph.

The rate of temperature rise of the magnetic particle in a given frequency and strength of AMF is related to the average size, size distribution, and type of the magnetic particle. Equivalently, for a magnetic particle of given average size and size distribution, the rate of heating is determined by the frequency and strength of AMF. Usually there is an optimal frequency that heats the magnetic particle most effectively. For most magnetic particles utilized in hyperthermia applications, the frequency of the AMF is in the range of 50 KHz-2 MHz. Note that in hyperthermia applications, the AMF is continuously applied, usually over 10 s of minutes.

In one embodiment, a method of the present disclosure includes generating thermo-acoustic signals for thermo-acoustic tomography. The method utilizes a magnetic field generator 404 to apply an amplitude-modulated alternating-magnetic-field (inset 408) to a magnetic particle contained in a portion of tissue 406 contained, that may be contained within a larger mass 402. The amplitude-modulated (e.g., pulsed) AMF 408, generates time varying heating (e.g., pulsed heating), which in turn produces an acoustic signal 410, that may be detected by sonic transducers 412.

Magnetic particles have been used as a contrast agent in TAT, under pulsed microwave excitation. The current method, in various embodiments, is different from such prior art in at least two aspects. A magnetic field is used instead of microwave. The frequency is also in the MHz range frequency versus the GHz range. The tissue attenuation of AMF is more than an order lower than that of microwave or light; therefore the illumination of tissue along the depth by AMF is significantly more uniform than that by light or microwave.

An amplitude-modulated (such as a pulse-enveloped) alternating magnetic field is used in the present embodiment instead of a pulsed magnetic field. It is noted that the magnetic field within the pulse duration of a pulse-enveloped alternating magnetic field alternates, in comparison to a non-alternating magnetic field within the pulse duration of a pulsed magnetic field. The mechanism of generating acoustic signal in the present embodiment is by heating using magnetic relaxation and cooling the magnetic particles rapidly to convert the thermal-energy to acoustic energy.

According to embodiments of the present disclosure, the magnetic-field device 404 used to generate the pulsed AMF can also be used to generate a conventional AMF to steadily heat the magnetic particle for hyperthermia. Thus the same magnetic particle(s) can be employed in both thermo-acoustic tomography and hyperthermia treatment.

Herein below is discussed the general heating function of magnetic nanoparticles under an amplitude-modulated multi-component alternating magnetic field. The conventional treatment of magnetic particle under a constant AMF has been revised to take into account the case of pulsed AMF heating according to the present disclosure.

An alternating magnetic field, with its amplitude modulated by an envelope, may be expressed by

$$H ( t ) = \{ {\sum }_{m = 1}^{M} {H}_{m} cos ( 2 π {f}_{m} t ) \} \cdot Ω ( t ) , \qquad ( 1 )$$

Where Hₘ is the amplitude of the magnetic field component with frequency fₘ, and Ω(t) is the envelope of the ensemble of all frequency components of the alternating magnetic field. So,
If M=1 and Ω(t)=1, equ (1) represents a sinusoidal AMF used for conventional magnetic hyperthermia;
If M>1 and Ω(t)=1, equ (1) represents an AMF with multiple frequency components, each generating independent heating for a linear magnetic system;
If M=1 and Ω(t)=func(t), equ (1) represents a sinusoidal AMF whose amplitude has been modulated by the function Ω(t), such as a low duty cycle pulse function; and
If M>1 and Ω(t)=func(t), equ (1) represents an AMF with multiple frequency components, and the amplitude of the ensemble has been modulated by the function Ω(t).

In this disclosure, we consider the cases of M=1, which is

H(t)=H cos(2πfₘt)·Ω(t) (2)

where the subscript m is now used to denote the single sinusoidal component of the “magnetic” field, and

$$Ω ( t ) = {\sum }_{n = 0}^{∞} [ u ( t - {nT}_{pulse\_duration} ) - u ( t - {t}_{pulse\_width} - {nT}_{pulse\_duration} ) ] \qquad ( 3 )$$

Where Tₚᵤₗₛₑ_{_{—}}_{duration}>>tₚᵤₗₛₑ_{_{—}}_{width}, and u(t) is the unit step function, or Heaviside function. The AMF represented by (2) and (3) is a sinusoidal AMF Hₘ cos(2#fₘt) turned on and off at the duty cycle defined by the unit pulse train of (3).

Because the AMF represented by (2) and (3) is a sinusoidal AMF Hₘ cos(2πfₘt) being turned on and off at the duty cycle defined by the unit pulse train of (3), the specific-loss-power (SLP) of the represented spatially-uniform AMF can be expressed by

S̅L̅P̅(\vec{r},t,Hₘ,fₘ)=μ₀πχ″(\vec{r},fₘ)Hₘ²fₘ/ρ·[Ω(t)]² (4)

Where μ₀=4π×10⁻⁷ VsA⁻¹m⁻¹, ρ is the mass density, fₘ is the frequency of the magnetic field. In a simple relaxation models, an assumption of an exponential decay of the magnetization with a relaxation time τ_{R} is given. For a linear system that is equivalent to a frequency spectrum χ″(\vec{r}, fₘ) of the type

$${χ}^{″} ( {r}^{->} , {f}_{m} ) = {χ}_{0} \frac{2 π {f}_{m} {τ}_{R} ( {r}^{->} )}{1 + {[ 2 π {f}_{m} {τ}_{R} ( {r}^{->} ) ]}^{2}} \\ {χ}_{0} = constant \qquad ( 5 )$$

Under the consideration of Neel relaxation and Brown relaxation

$$\begin{matrix}\frac{1}{{τ}_{R} ( {r}^{->} )} = \frac{1}{{τ}_{N} ( {r}^{->} )} + \frac{1}{{τ}_{B} ( {r}^{->} )} \\ And & ( 6 ) \\ {τ}_{N} ( {r}^{->} ) = {τ}_{0} exp [ \frac{K ( {r}^{->} ) \cdot V}{κ T} ] \\ {τ}_{0} \sim {10}^{- 9} s & ( 7 )\end{matrix}$$

Where K(\vec{r}) is the local anisotropy energy density, V is the particle volume or

$$V = \frac{π}{6} {d}^{3}$$

with d the diameter of the particle, κ is the Boltzmann constant, and T is the temperature in Kelvin.

$${τ}_{B} ( {r}^{->} ) = πη ( {r}^{->} ) \frac{{d}_{h}^{3}}{2 kT} \qquad ( 8 )$$

Where η is the local viscosity of the fluid suspension, dₕ³ is the hydrodynamic diameter.

Adding the contribution of hysteresis loss to SLP in eq. (4) is also possible.

Herein is discussed the equation of thermo-acoustic propagation by pulsed AMF-heating of MNP. The conventional treatment of thermo-acoustic propagation has been revised to take into account the SLP of MNP as the source of acoustic signal.

The equation of thermo-acoustic propogation is

$${∇}^{2} p ( {r}^{->} , t , {H}_{m} , {f}_{m} ) - \frac{1}{{v}_{s}^{2}} \frac{{\partial }^{2}}{\partial {t}^{2}} p ( {r}^{->} , t , {H}_{m} , {f}_{m} ) = - \frac{β}{{C}_{P}} \frac{\partial }{\partial t} \overline{S L P} ( {r}^{->} , t , {H}_{m} , {f}_{m} ) \qquad ( 9 )$$

Where p(\vec{r},t,Hₘ,fₘ) is the acoustic pressure, υₛ is the speed of sound, β is the isobaric volume expansion coefficient, Cₚ is the specific heat, S̅L̅P̅(\vec{r},t,Hₘ,fₘ) is the specific loss power representing the thermal energy per time and volume generated by the alternating magnetic field Hₘ at frequency fₘ.

We are initially interested in tissue with inhomogeneous AMF-absorption (due to the localized distribution of MNP) but a relatively homogenous acoustic property.

The solution of (1) based on Green's function can be found in the literature of physics or mathematics [12, 14]. A general form can be expressed as

$$p ( {r}^{->} , t , {H}_{m} , {f}_{m} ) = \frac{β}{4 π {C}_{P}} \int \int \int \frac{{d}^{3} {r}^{′}}{\vert {r}^{->} - {r}^{->} \vert} \frac{\partial \overline{S L P} ( {{r}^{->}}^{′} , {t}^{′} , {H}_{m} , {f}_{m} )}{\partial {t}^{′}} {\vert}_{{t}^{′} = t - ( \vert {r}^{->} - {{r}^{->}}^{′} \vert ) / {v}_{s}} \qquad ( 10 )$$

The SLP function can be written as the product of a spatial AMF absorption function (which is the distribution of MNP) and a temporal activation function of the AMF field

S̅L̅P̅(\vec{r},t,Hₘ,fₘ)=A(\vec{r},Hₘ,fₘ)η(t)={μ₀πχ″(\vec{r},fₘ)Hₘ²fₘ/ρ}·[Ω(t)]² (11)

Thus, p(\vec{r},t,Hₘ,fₘ) can be expressed as

$$\begin{matrix}p ( \vec{r} , t , {H}_{m} , {f}_{m} ) = \frac{β}{4 π {C}_{P}} \int \int \int \frac{{d}^{3} {r}^{′}}{\vert \vec{r} - {\vec{r}}^{′} \vert} A ( {\vec{r}}^{′} , {H}_{m} , {f}_{m} ) \frac{d η ( {t}^{′} )}{d {t}^{′}} {\vert}_{{t}^{′} = t - ( \vert \vec{r} - {\vec{r}}^{′} \vert ) / {υ}_{s}} = \frac{β}{4 π {C}_{P}} \int \int \int \frac{{d}^{3} {r}^{′}}{\vert \vec{r} - {\vec{r}}^{′} \vert} \{ {μ}_{0} π {χ}^{″} ( {\vec{r}}^{′} , {f}_{m} ) {H}_{m}^{2} {f}_{m} / ρ \} \frac{d η ( {t}^{′} )}{d {t}^{′}} {\vert}_{{t}^{′} = t - ( \vert \vec{r} - {\vec{r}}^{′} \vert ) / {υ}_{s}} = \frac{β {μ}_{0} {χ}_{0}}{4 ρ {C}_{P}} {H}_{m}^{2} {f}_{m} \int \int \int \frac{{d}^{3} {r}^{′}}{\vert \vec{r} - {\vec{r}}^{′} \vert} \{ \frac{2 π {f}_{m} {τ}_{R} ( {\vec{r}}^{′} )}{1 + {[ 2 π {f}_{m} {τ}_{R} ( {\vec{r}}^{′} ) ]}^{2}} \} \frac{d η ( {t}^{′} )}{d {t}^{′}} {\vert}_{{t}^{′} = t - ( \vert \vec{r} - {\vec{r}}^{′} \vert ) / {υ}_{s}} \\ Denote & ( 12 ) \\ ψ ( {H}_{m} , {f}_{m} ) = \frac{β {μ}_{0} {χ}_{0}}{4 ρ {C}_{P}} & ( 13 ) \\ ϕ ( {\vec{r}}^{′} ) = \frac{2 π {f}_{m} {τ}_{R} ( {\vec{r}}^{′} )}{1 + {[ 2 π {f}_{m} {τ}_{R} ( {\vec{r}}^{′} ) ]}^{2}} & ( 14 )\end{matrix}$$

Equation (12) becomes

$$\begin{matrix}p ( \vec{r} , k , {H}_{m} , {f}_{m} ) = ψ ( {H}_{m} , {f}_{m} ) \int \int \int \frac{{d}^{3} {r}^{′}}{\vert \vec{r} - {\vec{r}}^{′} \vert} ϕ ( {r}^{′} ) \frac{d η ( {t}^{′} )}{d {t}^{′}} {\vert}_{{t}^{′} = t - ( \vert \vec{r} - {\vec{r}}^{′} \vert ) / {υ}_{s}} \\ = ψ ( {H}_{m} , {f}_{m} ) \int \int \int ϕ ( {r}^{′} ) \frac{d η ( {t}^{′} )}{d {t}^{′}} {\vert}_{{t}^{′} = t - ( \vert \vec{r} - {\vec{r}}^{′} \vert ) / {υ}_{s}} \frac{{d}^{3} {r}^{′}}{\vert \vec{r} - {\vec{r}}^{′} \vert}\end{matrix} \qquad ( 15 )$$

For constant amplitude, dI/dt=0, so constant rate of heating does not induce acoustic pressure.

We proceed by transforming the time-depend wave equation into the temporal-frequency domain. Denoting the Fourier transforms of p and η by p̅ and η̅, we have

p̅(\vec{r},k,Hₘ,fₘ)=∫_{-∞}^{∞}p(\vec{r},t,Hₘ,fₘ)exp(ikt)dt (16)

η̅(k)=∫_{-∞}^{∞}η(t)exp(ikt)dt (17)

Substituting (5) and (6) into (4) results in

$$p ( \vec{r} , k , {H}_{m} , {f}_{m} ) = {\int }_{- ∞}^{∞} p ( \vec{r} , k , {H}_{m} , {f}_{m} ) exp ( i kt ) d t = {\int }_{- ∞}^{∞} \{ ψ ( {H}_{m} , {f}_{m} ) \int \int \int ϕ ( {r}^{′} ) \frac{d η ( {t}^{′} )}{d {t}^{′}} \frac{{d}^{3} {r}^{′}}{\vert \vec{r} - {\vec{r}}^{′} \vert} \} exp ( i kt ) d t = i k \overline{η} ( k ) ψ ( {H}_{m} . {f}_{m} ) \int \int \int ϕ ( {r}^{′} ) \frac{exp ( i k \vert \vec{r} - {\vec{r}}^{′} \vert )}{\vert \vec{r} - {\vec{r}}^{′} \vert} {d}^{3} {r}^{′} \qquad ( 18 )$$

If the acoustic signals are collected along a line or in a plane, for example, at z=0, following the line of Nortan and Linze in, it can be shown that for the case of |k|>rho and z′>0

Sgn(k) is the signum function, ξ²=u²+v² (21)

$$p ( \vec{r} , k , {H}_{m} , {f}_{m} ) = i k \overline{η} ( k ) ψ ( {H}_{m} , {f}_{m} ) \int \int \int ϕ ( {r}^{′} ) \frac{exp ( i k \vert \vec{r} - {\vec{r}}^{′} \vert )}{\vert \vec{r} - {\vec{r}}^{′} \vert} {d}^{3} {r}^{′} \\ \overline{P} ( u , v , k , {H}_{m} , {f}_{m} ) = \frac{4 {π}^{2} k \overline{η} ( k ) ψ ( {H}_{m} , {f}_{m} )}{\sqrt{{k}^{2} - {ζ}^{2}}} {\int }_{0}^{∞} ϕ ( u , v , {z}^{′} ) exp [ - i {z}^{′} sgn ( k ) \sqrt{{k}^{2} - {ζ}^{2}} ] d {z}^{′} \qquad ( 22 )$$

The above equation can further be simplified to

$$\begin{matrix}\overline{P} ( u , v , k , {H}_{m} , {f}_{m} ) = \frac{4 {π}^{2} k \overline{η} ( k ) ψ ( {H}_{m} , {f}_{m} )}{\sqrt{{k}^{2} - {ζ}^{2}}} {Φ}_{1} [ u , v , sgn ( k ) \sqrt{{k}^{2} - {ζ}^{2}} ] \\ where & ( 23 ) \\ {Φ}_{1} ( u , v , w ) = \frac{1}{2 π} {\int }_{- ∞}^{∞} ϕ ( u , v , {z}^{′} ) exp ( i {wz}^{′} ) d {z}^{′} & ( 24 )\end{matrix}$$

An inverse Fourier transform of (22) leads to the exact reconstruction of the acoustic source.

Further to the methods of the present disclosure, as the size of MNP reaches the super-paramagnetic domain, Brownian relaxation and Néel relaxation become increasingly dominant in the heat dissipation process. By optimizing the AMF parameters according to the dimensional and material properties of the MNPs, high specific loss power (SLP) from the MNPs can be achieved. Highly efficient heating of MNPs using steady AMF, aided by localized or systematic targeting of MNPs to a disease site by conjugating MNPs with a ligand of biomarkers, has significantly enhanced the potential of hyperthermia for cancer treatment and enabled developments in controlled drug release.

In nearly all therapeutic applications of MNPs that utilize AMF to induce heat as the vehicle of treatment, the AMF is applied continuously over a duration that lasts typically a few tens of minutes. In some studies of controlled drug release the AMF may be applied at a subsequent, long-pulse mode. The AMF within each of the minutes-long pulses is effectively steady-state because the frequency of AMF is at least at KHz range.

Although the quantitative mechanism of AMF-induced heating of MNPs is still subject to discussion, most studies adopt Rosensweig's model to quantify the Brownian and Neel relaxation characteristics of MNPs as applied to AMF-induced heat dissipation. Rosensweig's model justified a strong dependence of the heating efficacy upon the frequency of AMF for a given MNP size-domain when the magnetic field intensity is below the threshold to saturate the magnetization.

For a mono-dispersed super-paramagnetic iron oxide nanoparticle (SPION), the model predicted relaxation peak is usually at or above 1 MHz. However, in most studies involving AMF-mediated heating of MNP, the AMF frequencies generally range between 100 to 500 KHz, and the field intensities range between 50 to 300 Oe. The diverse AMF parameters are due to the situation that most AMF devices used for individual studies were custom-developed but there also exists inconsistencies in safety concerns over the course of treatment if the product of the field intensity and frequency of AMF exceeds a perceived limit.

Time-varying AMF-mediated heating of MNPs can be achieved by either a time-domain or a frequency-domain AMF configuration. The time-domain AMF configuration refers to applying AMF over a short duration within which the AMF remains steady-state, and the frequency-domain AMF configuration refers to applying AMF continuously at fixed amplitude but with the frequency modulated (chirped). With a time-domain AMF, the heating of MNPs is to be established and then removed instantly following the application duty cycle of AMF. With a frequency-domain AMF, the heating of MNP varies following the cycle of frequency modulation of AMF as a result of the strong frequency dependence of heat dissipation of MNPs.

The simplest form of a time-domain AMF may be a short burst of AMF of which the duration is greater than (and for the convenience of analysis should contain integer number of) one period of the magnetic field oscillation. A magnetic field intensity that does not oscillate within the burst (but could vary over the duration of the burst) is simply a pulsatile magnetic field, which has been applied to magneto-acoustic modulation of MNPs for ultrasound imaging, magneto-motive optical coherence tomography, magneto-acoustic tomography with magnetic induction (MAT-MI) and magneto-acoustic tomography of MNPs. In all these approaches the effect of the pulsatile magnetic field upon MNPs is a translational mechanical force imposed by the spatial gradient of the magnetic field. The magneto-thereto-acoustic wave generation of the present disclosure results from applying time- or frequency-domain AMF upon MNPs resulting in a magnetic relaxation loss that converts magnetic field energy to heat. This is also mechanistically different from a dielectric loss of microwave energy in microwave-induced thermo-acoustics.

Below, Rosensweig's model is implemented in an alternative form to describe the heat dissipation of MNPs within one complete cycle (a 2π phase change) of AMF intensity oscillation. The heat dissipation of MNPs is derived within a short burst of AMF that contains integer numbers of complete cycles of AMF intensity oscillation and the heat dissipation of MNPs within each 2π phase change of a linearly frequency chirped AMF.

Rosensweig's model, by default, assumed a continuous-wave (CW) or steady-state AMF (i.e. the magnetic field intensity alternates at a fixed frequency and constant amplitude, and expressed the generated heat by volumetric power dissipation—the volumetric heat accumulated over one second—and it remains constant for a CW AMF over the course of magnetic field application). In the present embodiment, the AMF is applied at a short duration (e.g., micro-second scale) that may allow only a limited number of complete cycles of the magnetic field oscillation. To quantify the total heat dissipation over the micro-second burst-duration of applying AMF, one can either scale the volumetric heat dissipation from over one second to over the micro-second duration of the burst or equivalently multiply the volumetric heat generated over ONE cycle of the AMF oscillation with the NUMBER of cycles (assuming integer numbers for convenience) contained in the duration of the AMF burst. In the present case, as the AMF is to be applied continuously, but the frequency changes, the heat dissipation imposed has to be quantified for each individual cycle of the AMF field oscillation.

To facilitate the quantifications of the heat dissipation by MNPs in time-domain and frequency-domain AMF configurations, Rosensweig's model is used in an alternative form to represent the volumetric heat dissipation over a 2π phase change of a steady-state AMF. The result is used as the base formula to analyze the heat dissipation of MNPs accumulated over the bursting duration of an AMF in time-domain configuration, and to compare it with the time-varying heat dissipation of MNPs over each individual cycles of a frequency-chirped AMF. Notice that photo-acoustics has already established the relation between the heat-dissipation and the initial acoustic pressure of the thermally induced acoustic wave, under the condition that the irradiation time-scale satisfies thermal and acoustic confinement, and that tissue-attenuation of magnetic field is negligibly small compared to that of light. Therefore the feasibility of magneto-thermo-acoustics can be evaluated by comparing the heat dissipation of MNPs when exposed to a time-domain or a frequency-domain AMF of practical utility against the heat dissipation by a chromophore at different tissue-depths when irradiate by the maximum surface light flence in photo-acoustics.

We adapt Rosensweig's model to derive the heat dissipation by MNPs over a 2π phase change of a steady-state AMF. Assuming a constant density system, the first law of thermodynamics governs that

$$\frac{d U}{d t} = \frac{d Q}{d t} + \frac{d W}{d t} \qquad ( 2.1 )$$

where U [unit: J] is the internal energy, Q [unit: J] is the heat added, and W [unit: J] is the magnetic work done on the system. The differential magnetic work by a collinear magnetic field is dW=\vec{H}·d\vec{B}=H·dB, where \vec{H} [unit: A m⁻¹ or 4π×10⁻³ Oe] is the magnetic field intensity and \vec{B} [unit: T or V s A⁻¹ m⁻²] is the magnetic induction. As B=μ₀(H+M), where M [unit: A m⁻¹] is the magnetization and μ₀=4π×10⁻⁷ [unit: V s A⁻¹ m⁻¹] is the permeability of free space, the differential internal energy for an adiabatic process, i.e. ∂Q=0, becomes

$$\frac{d U}{d t} = {μ}_{0} H \cdot ( \frac{d H}{d t} + \frac{d M}{d t} ) \qquad ( 2.2 )$$

Denoting the dimension-less complex magnetic susceptibility of MNPs as χ=χ′−iχ″, the real part of the susceptibility and the imaginary part of the susceptibility χ″ under a time-varying magnetic field with an instant angular frequency ω become respectively

$$\begin{matrix}\{ \begin{matrix}{χ}^{′} ( ω ) = {χ}_{0} \frac{1}{1 + {[ ω {τ}_{R} ]}^{2}} \\ {χ}^{″} ( ω ) = {χ}_{0} \frac{ω {τ}_{R}}{1 + {[ ω {τ}_{R} ]}^{2}}\end{matrix} & ( 2.3 )\end{matrix}$$

where τ_{R} [unit: s] is the relaxation time, and χ₀ is the equilibrium susceptibility which can be calculated from the following expressions:

$$\begin{matrix}{χ}_{0} = {χ}_{i} \frac{3}{ξ} ( coth ξ - \frac{1}{ξ} ) \\ where & ( 2.4 . a ) \\ {χ}_{i} = \frac{{μ}_{0} φ {M}_{d}^{2} {V}_{M}}{3 {k}_{B} {T}_{emp}} & ( 2.4 . b ) \\ ξ = \frac{{μ}_{0} {M}_{d} {H}_{0} {V}_{M}}{{k}_{B} {T}_{emp}} & ( 2.4 . c )\end{matrix}$$

where φ [dimensionless] is the volume fraction of the MNP solid in the host liquid matrix, M_{d} [unit: A m⁻¹] is the domain magnetization of MNP, V_{M} [unit: m³] is the magnetic volume of MNP, H₀ is the amplitude of the magnetic field intensity, k_{B}=1.38×10⁻²³ [unit: m² kg s⁻² K⁻¹] is the Boltzmann constant, and Tₑₘₚ [unit: K] is the temperature. If the MNP in a liquid matrix is mono-dispersed in the super-paramagnetic-size domain, the relaxation time τ_{R} is to be dominated by Néel and Brownian relaxations as:

$$\frac{1}{{τ}_{R}} = \frac{1}{{τ}_{N}} + \frac{1}{{τ}_{B}} \qquad ( 2.5 )$$

The Néel relaxation time τ_{N} in Eq. (2.5) is:

$${τ}_{N} = \frac{\sqrt{π}}{2} {τ}_{0} \frac{exp ( \frac{κ \cdot {V}_{M}}{{k}_{B} {T}_{emp}} )}{\sqrt{\frac{κ \cdot {V}_{M}}{{k}_{B} {T}_{emp}}}} {τ}_{0} \sim {10}^{- 9} s \qquad ( 2.6 . N )$$

where κ [unit: J m⁻³] is the anisotropy energy density, and τ₀ is a nanosecond-scale characteristic time. The Brownian relaxation time τ_{B} in Eq. (2.5) is:

$${τ}_{B} = \frac{3 η \cdot {V}_{H}}{{k}_{B} {T}_{emp}} \qquad ( 2.6 . B )$$

where V_{H} [unit: m³] is the hydrodynamic volume of MNP, and η [unit: N s m⁻²] is the viscosity coefficient of the matric fluid.

A steady-state or CW AMF is represented by

H(t)=H₀ cos(ω₀t)= [symbol] [H₀exp(iω₀t)] (2.7.CW)

under which the MNP magnetization is

M(t)= [symbol] [χ·H₀exp(iω₀t)]=H₀[χ′·cos(ω₀t)+χ″·sin(ω₀t)] (2.8.CW)

then Eq. (2.2) becomes

$$\frac{d U}{d t} = \frac{1}{2} {μ}_{0} {ω}_{0} {H}_{0}^{2} [ - ( 1 + {χ}^{′} ) \cdot sin ( 2 {ω}_{0} t ) + {χ}^{″} \cdot cos ( 2 {ω}_{0} t ) + {χ}^{″} ] \qquad ( 2.9 . CW )$$

Integrating Eq. (2.9.CW) over a full cycle or 2π phase change of AMF oscillation results in the heat dissipation per unit volume [unit: J m⁻³] over a duration of

$$Δ {t}_{2 π} = \frac{2 π}{{ω}_{0}} as \\ \begin{matrix}Δ {q}_{2 π} = {\int }_{t - Δ {t}_{2 π}}^{t} {U}^{′} d t \\ = {μ}_{0} π {H}_{0}^{2} {χ}^{″} \\ = {μ}_{0} π {H}_{0}^{2} {χ}_{0} \frac{{ω}_{0} {τ}_{R}}{1 + {[ {ω}_{0} \cdot {τ}_{R} ]}^{2}}\end{matrix} \qquad ( 2.10 . CW )$$

The thermal energy deposited per unit volume per unit time, i.e. the volumetric power dissipation [unit: W m^{×3}], is then

$${q}_{CW} = Δ {q}_{2 π} \cdot \frac{1}{Δ {t}_{2 π}} = \frac{{μ}_{0} {χ}_{0}}{2 {τ}_{R}} \frac{{[ {ω}_{0} \cdot {τ}_{R} ]}^{2}}{1 + {[ {ω}_{0} \cdot {τ}_{R} ]}^{2}} {H}_{0}^{2} \qquad ( 2.11 )$$

where the subscript “CW” denotes “continuous-wave”. Accordingly, for a MNP-liquid system that has a mass-density ρ [unit: kg m⁻³] the specific-loss-power (SLP) [unit: W kg⁻¹] is

$${SLP}_{CW} = \frac{{q}_{CW}}{ρ} = \frac{{μ}_{0} {𝒳}_{0}}{2 {ρτ}_{R}} \frac{{[ {ω}_{0} \cdot {τ}_{R} ]}^{2}}{1 + {[ {ω}_{0} \cdot {τ}_{R} ]}^{2}} {H}_{0}^{2} \qquad ( 2.12 )$$

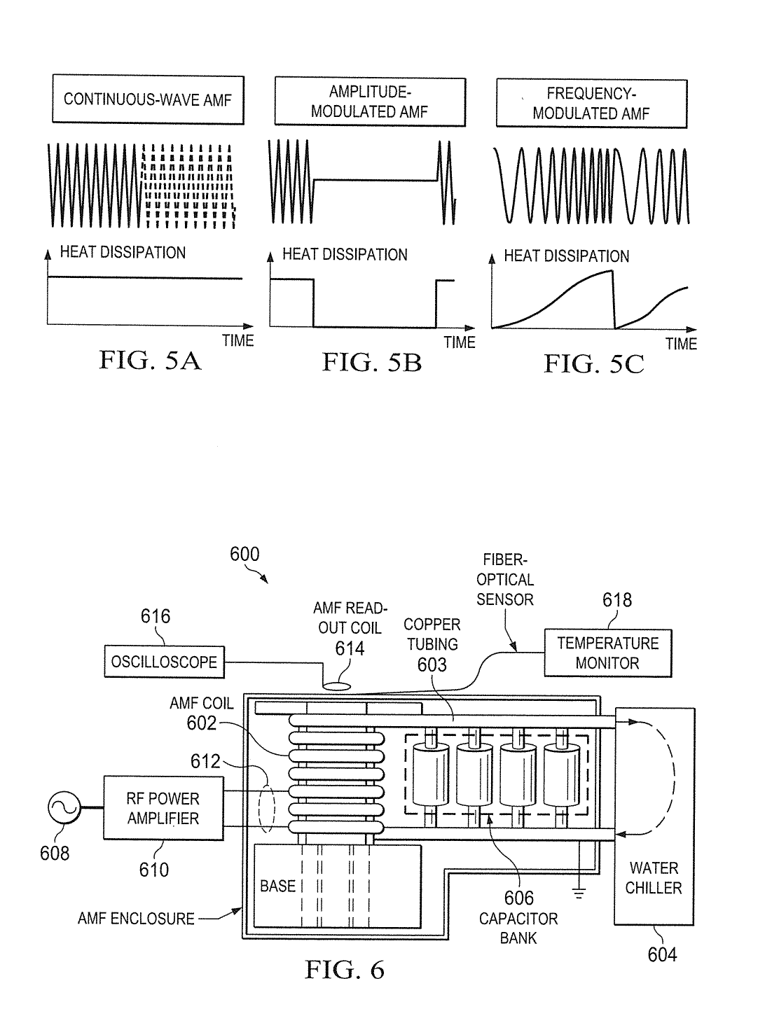

Under a CW AMF as illustrated in FIG. 5(A), the heat is continuously deposited by MNP at a constant rate as specified by Eq. (2.12), therefore no thermo-acoustic wave is to be generated, and the effect of this time-invariant heat dissipation is a steady rise of the local temperature for an adiabatic process. In practice, however, the MNP-liquid matrix transfers the heat to the ambient environment, so the temperature and volume of MNP-liquid matrix will rise and expand until a thermo-equilibrium with the environment is reached. The initial rate of the temperature rise of the MNP-liquid system is defined as [10]:

$$\frac{d {T}_{emp}}{d t} {\vert}_{t = 0} = \frac{{SLP}_{CW}}{{C}_{V}} = \frac{{μ}_{0} {𝒳}_{0}}{2 ρ {C}_{V} {τ}_{R}} \frac{{[ {ω}_{0} \cdot {τ}_{R} ]}^{2}}{1 + {[ {ω}_{0} \cdot {τ}_{R} ]}^{2}} {H}_{0}^{2} \qquad ( 2.13 )$$

where Cᵥ [unit: J kg⁻¹ K⁻¹] is the specific heat of the MNP-liquid system at a constant volume. Equation (2.13) is frequently used to predict and experimentally deduce the SLP of MNPs when exposed to a steady-state AMF for studies of localized hyperthermia and controlled drug release.

By exposing MNPs to an AMF of a short duration, such as a micro-second burst within which the magnetic field intensity of AMF alternates at several MHz, the relaxation of MNP will be established abruptly as the AMF burst is turned ON and removed instantaneously as the AMF burst is turned OFF. The abrupt onset and removal of the AMF will result in rapidly time-varying heat dissipation, as depicted in FIG. 5(B), which in turn will induce thermo-acoustic wave generation.

From the derivation of Eq. (2.10), it is appreciated that the cumulative contribution of the real part of the magnetic susceptibility to the internal energy of MNP over a phase change of a steady-state AMF is zero. Therefore as long as there are integer numbers of phase change (or equivalently integer number of complete cycles of oscillation) of the magnetic field within a short duration of applying the field, of Eq. (2.10) still quantifies the heat dissipation per unit volume over each single phase change of the AMF. Consequently multiplying with the total number of complete cycles of magnetic field oscillation gives the total heat dissipation per unit volume that is accumulated over the duration of AMF application. In terms of the width of the bursting of AMF for imaging purposes, as the spatial resolution of thermo-acoustics is bounded by the length of acoustic propagation in biological medium during the onset of heat dissipation, a burst width of AMF less than 1 is needed if the axial resolution of acoustic detection is to be better than 1.55 mm. A pulse width of 1 μs is common to the microwave-irradiation in microwave-induced thermo-acoustic tomography, though much longer than the pulse width of light irradiation in photo-acoustics.

We thus consider a simplest form of time-domain AMF, as illustrated in FIG. 5(B), a short burst of fixed frequency and fixed amplitude AMF applied repetitively at a low duty cycle. This short bursting of AMF can be expressed as a “carrier” AMF being modulated by an envelope function of a pulse train. The envelope function, denoted by Ω(t), is written by using the Heaviside or unit-step function u(t) as

$$Ω ( t ) = {\sum }_{n = 0}^{∞} [ u ( t - n \cdot Δ {T}_{TD} ) - u ( t - Δ {t}_{ON} - n \cdot Δ {T}_{TD} ) ] ; \\ n = 1 , 2 , 3 , \qquad ( 2.14 )$$

where ΔT_{T} is the period of the pulse train, the subscript “TD” denotes “time-domain”, and Δt_{ON} is with of each burst within which the AMF at a fixed frequency and fixed amplitude is applied. The time sequence Ω(t) of Eq. (2.14) basically specifies when a steady-state AMF is turned ON or OFF, and it satisfies the following specific condition

Ω(t)=[Ω(t)]² (2.14.*)

without which the following Eq. (2.10.TD) should contain additional terms. The magnetic field of this time-domain AMF is then represented by

H_{TD}(t)=H₀(t)cos(ω₀t)=Ω(t)[H₀ cos(ω₀t)] (2.7.TD)

Based on Eq. (2.10) the volumetric heat dissipation of MNPs at a position \vec{r}′ due to a pulse-enveloped time-domain AMF characterized by Eq. (2.7TD), (2.14) and (2.14*) is

$$Δ {q}_{{ }_{TD}} ( {{r}^{->}}^{′} , t ) = {μ}_{0} π {H}_{0}^{2} {𝒳}_{0} ( {{r}^{->}}^{′} ) \frac{{ω}_{0} {τ}_{R} ( {{r}^{->}}^{′} )}{1 + {[ {ω}_{0} {τ}_{R} ( {{r}^{->}}^{′} ) ]}^{2}} \frac{Δ {t}_{ON}}{Δ {t}_{2 π}} \qquad ( 2.10 . TD )$$

Following the time-varying cycle of Ω(t), the heat dissipation q_{TD} (\vec{r}′,t) of MNP that varies rapidly over time will give rise to a thermo-acoustic wave, at the rising and falling edges of Ω(t), Notice that Eq. (2.10.TD) is derived by assuming that the steady heat dissipation is established at an infinitesimally short moment after turning ON the steady-state AMF and removed immediately after turning OFF the steady-state AMF, according to Eq. (2.14). Such assumption ignores the effect of high frequency components of the AMF arising due to the finite time-scale of establishing or removing the AMF field, which in reality will complicate the signal spectrum of thermo-acoustic wave.

The thermally generated acoustic pressure p_{TD}(\vec{r},t) at a specific location \vec{r} satisfies the following equation that has been well documented in photo- or microwave-induced thermo-acoustics:

$${∇}^{2} {p}_{TD} ( {r}^{->} , t ) - \frac{1}{{c}_{a}} \frac{{\partial }^{2}}{\partial {t}^{2}} {p}_{TD} ( {r}^{->} , t ) = - \frac{β}{{C}_{p}} \frac{\partial }{\partial t} Δ {q}_{TD} ( {r}^{->} , t ) \qquad ( 2.15 . TD )$$

where cₐ [unit: m s⁻¹] is the speed of acoustic wave in tissue, β [unit: K⁻¹] is the isobaric volume thermal expansion coefficient, and Cₚ [unit: J kg⁻¹K⁻¹] is the specific heat at a constant pressure. The general solution of the acoustic pressure originating from the source of thermo-acoustic wave at \vec{r}′ and reaching a point transducer at \vec{r} in an unbounded medium is:

$${p}_{TD} ( {r}^{->} , t ) = \frac{β}{4 π {C}_{p}} {\int }_{v} \frac{1}{\vert {r}^{->} - {{r}^{->}}^{′} \vert} \frac{\partial }{\partial t} Δ {q}_{TD} ( {{r}^{->}}^{′} , t - \frac{\vert {r}^{->} - {{r}^{->}}^{′} \vert}{{c}_{a}} ) \cdot {d}^{3} {{r}^{->}}^{′} \qquad ( 2.16 . TD )$$

When the distance between the source and the measurement points, l=|\vec{r}−\vec{r}′|, is much greater than the dimension of the source, and the thermo-acoustic source is approximated by a uniform distribution of MNPs in a volume V(\vec{r}′), Eq. (2.16.TD) can be simplified to

$${P}_{TD} ( {r}^{->} , t ) = \frac{β}{4 π {C}_{p}} \frac{V ( {{r}^{->}}^{′} )}{l} \frac{\partial }{\partial t} Δ {q}_{TD} ( {{r}^{->}}^{′} , t - \frac{l}{{c}_{a}} ) \qquad ( 2.17 . TD )$$

Equation (2.15.TD) states that time-invariant heat dissipation does not induce thermo-acoustic wave, which is what occurs when CW AMF of fixed frequency and amplitude is applied upon MNPs. However, thermo-acoustic wave generation could have occurred at the instants of setting ON and setting OFF the CW AMF, were the rising and falling edges of the steady-state AMF very rapid in hyperthermia and particularly in the studies of triggered drug release wherein the minute-long AMF trains were repetitively applied.

The simplest form of a frequency-domain AMF may be one with linearly modulated or chirped frequency, as shown in FIG. 5(C), which has an instantaneous angular frequency of

ω(t)=ωₛₜ+bt (2.18)

where ωₛₜ is the starting frequency and b is the rate of frequency sweeping. The instantaneous field strength of this linearly frequency chirped AMF is

H_{FD}(t)=H₀ cos [ωt]=H₀ cos [(ωₛₜ+bt)t] (2.7.FD)

where the subscript “FD” denotes “frequency-domain”. The resulted magnetization is

M(t)= [symbol] [χ·H₀exp(iωt)]=H₀[χ′(ω)·cos(ωt)+χ″(ω)·sin(ωt)] (2.8.FD)

Substituting Eqs. (2.7.FD) and (2.8.FD) to Eq. (2) leads to

$$\frac{d U}{d t} = \frac{1}{2} {μ}_{0} {H}_{0}^{2} [ ( 1 + cos ( 2 ω t ) ) \frac{d {𝒳}^{′}}{d t} + sin ( 2 ω t ) \frac{d {𝒳}^{″}}{d t} + {𝒳}^{″} ( 1 + cos ( 2 ω t ) ) \frac{d ( ω t )}{d t} - ( 1 + {𝒳}^{′} ) sin ( 2 ω t ) \frac{d ( ω t )}{d t} ] \qquad ( 2.9 . FD )$$

We denote a “positive-zero-crossing” phase as the instant when the magnetic field strength is zero and the next value is positive, i.e. the instant that crosses the abscissas upwardly. Then integrating Eq. (2.9.FD) over a 2π phase change of the AMF starting at a “positive-zero-crossing” phase is equivalent to integrating Eq. (2.9.FD) from an earlier phase of ω₀t₀=(n−1)*2π, where n is a positive integer, to the current phase of ωt=n*2π. If we denote Δt_{2π} as the time taken for the phase of AMF to change 2π from the earlier “positive-zero-crossing” instant to the current “positive-zero-crossing” instant, we have

$${ω}_{0} {t}_{0} = {ω}_{0} ( t - Δ {t}_{2 π} ) = ω t - 2 π \\ or \\ Δ {t}_{2 π} = \frac{2 π}{{ω}_{0}} - \frac{( ω - {ω}_{0} )}{{ω}_{0}} t \qquad ( 2.19 )$$

and the integration of Eq. (2.9.FD) over Δt₂, duration results in the following instantaneous volumetric heat dissipation

$$Δ {q}_{FD} ( t ) = {\int }_{t - Δ {t}_{2 π}}^{t} {U}^{′} d t = {μ}_{0} π {H}_{0}^{2} {𝒳}_{0} \frac{{ωτ}_{R}}{1 + {[ ω \cdot {τ}_{R} ]}^{2}} - \frac{5}{4} {μ}_{0} {H}_{0}^{2} {𝒳}_{0} [ \frac{1}{1 + {( {ω}_{0} \cdot {τ}_{R} )}^{2}} - \frac{1}{1 + {( ω \cdot {τ}_{R} )}^{2}} ] \qquad ( 2.10 . FD )$$

Apparently Eq. (2.10.FD) becomes Eq. (2.10.CW) for CW AMF if the frequency modulation is turned off (i.e. b=0 in Eq. (2.18)). With the frequency modulation, Δq_{2π}(t) represented by Eq. (2.10.FD) changes periodically following the cycle of the frequency chirping, and the instantaneous Δq_{2π}(t) is strongly dependent upon the AMF frequency according to the magnetic susceptibility term at a given magnetic field intensity. Notice that the second term in Eq. (2.10.FD) that involves the differentiation between the earlier “positive-zero-crossing” phase and the current “positive-zero-crossing” phase will modify the proportionality of the heat dissipation to the first term in Eq. (2.10.FD). Collectively, the time-varying heat dissipation upon MNPs due to frequency-chirped AMF mediation will give rise to a thermo-acoustic wave.

We denote Δq_{FD}(\vec{r},t) as the volumetric heat dissipation at a position \vec{r} at an instant t due to a frequency chirped AMF represented by Eq. (2.7.FD), and the Fourier transform of Δq_{FD}(\vec{r},t) as ΔQ_{FD}(\vec{r},\tilde{ω}). Accordingly, the acoustic pressure excited by Δq_{FD}(\vec{r},t) is represented by p_{FD}(\vec{r},t), and the Fourier transform of p_{FD}(\vec{r},t) is denoted as P_{FD}(\vec{r},\tilde{ω}). The propagation of \tilde{P}_{FD}(\vec{r},\tilde{ω}) then satisfies the following Fourier-domain wave equation

$${∇}^{2} {\tilde{P}}_{FD} ( {r}^{->} , \tilde{ω} ) + \frac{{( \tilde{ω} )}^{2}}{{c}_{a}} {\tilde{P}}_{FD} ( {r}^{->} , \tilde{ω} ) = - \frac{i \tilde{ω} β}{{C}_{p}} Δ {\tilde{Q}}_{FD} ( {r}^{->} , \tilde{ω} ) \qquad ( 2.15 . FD )$$

The general solution of Eq. (2.15.FD) for the acoustic pressure reaching a transducer at \vec{r} and originating from the source of thermo-acoustic wave at \vec{r}′ in an unbounded medium is [26]

$${\tilde{P}}_{FD} ( {r}^{->} , \tilde{ω} ) = - \frac{i \tilde{ω} β}{4 π {C}_{p}} {\int }_{V} \frac{exp ⌊ i k \vert {r}^{->} - {{r}^{->}}^{′} \vert ⌋}{\vert {r}^{->} - {{r}^{->}}^{′} \vert} Δ {\tilde{ Q}}_{FD} ( {r}^{->} , \tilde{ω} ) {d}^{3} {{r}^{->}}^{′} \qquad ( 2.20 )$$

If the distance between the source and the measurement points, l=|\vec{r}−\vec{r}′|, is much greater than the dimension of the source, and that the thermo-acoustic source is approximated by a uniform distribution of MNPs in a volume V(\vec{r}′), Eq. (2.20) is simplified to

$${\tilde{P}}_{FD} ( {r}^{->} , \tilde{ω} ) = - \frac{i \tilde{ω} β}{4 π {C}_{p}} \frac{V ( {{r}^{->}}^{′} )}{l} Δ {\tilde{ Q}}_{FD} ( {r}^{->} , \tilde{ω} ) exp [ i k \vert {r}^{->} - {{r}^{->}}^{′} \vert ] \qquad ( 2.16 . FD )$$

so the acoustic wave intercepted by a point ultrasound transducer at \vec{r} that locates at a distance of l=|\vec{r}−\vec{r}′| from the source of thermo-acoustic wave can be written as

$${P}_{FD} ( {r}^{->} , t ) = \frac{β}{4 π {C}_{p}} \frac{V ( {{r}^{->}}^{′} )}{l} \frac{\partial }{\partial t} Δ {q}_{FD} ( {r}^{′} , t - \frac{l}{{c}_{a}} ) exp \{ i [ \tilde{ω} ( t - \frac{l}{{c}_{a}} ) + {θ}_{a} ] \} \qquad ( 2.17 . FD )$$

where θₐ is a phase constant related to thermo-elastic conversion. Equation (2.17.FD) states that a frequency invariant AMF mediation, as it gives rise to a constant Δq, does not induce thermo-acoustic wave upon MNP.

We estimate the heat dissipation of a SPION sample in a time-domain or frequency-domain configuration of AMF at 100 Oe field intensity that may be of practical utility, by comparison to the heat dissipation due to chromophore at different depths in a typical biological tissue when subjected to ANSI limited surface irradiation fluence of near-infrared light for non-therapeutic use. The estimation is rendered by experimentally measured heating characteristics of a SPION sample of 0.8 mg/ml iron-weight concentration when exposed to CW AMF of various frequencies ranging from 88.8 KHz to 1.105 MHz and normalized at 100 Oe field intensity. The experimentally measured heating characteristics are modeled by Eq. (2.13), and the model is extrapolated to 10 MHz in order to evaluate the potential of magneto-thermal heat dissipation by 10 complete cycles of AMF oscillation within a 1-μs burst, the width necessary to achieving a 1.55 mm axial resolution of acoustic detection. In comparison, the volumetric heat dissipation by a 100 mJ/cm² near-infrared surface illumination upon a chromophore that has 1 fold or 10 folds of absorption contrast over the background biological medium that has a reduced scattering coefficient of 10 cm⁻¹ and an absorption coefficient of 0.1 cm⁻¹ is evaluated. The time-varying volumetric heat dissipation by the SPION sample exposed to an AMF train that chirps linearly from 1 MHz to 10 MHz over a 1 ms duration is also estimated.

For some embodiments of the present disclosure, utilizing SPION a pulse-enveloped alternating magnetic field may be expected to work well when the frequency of the alternating magnetic field (when such magnetic field is active or on) is at or above 10 MHz. At this frequency super-paramagnetic iron oxide nanoparticles usually have saturated (maximum) heating power, which would allow the thermo-acoustic wave generation to be more efficient. The duration of the pulse-enveloped alternating magnetic field to be active (i.e. when the field is ON) may be at or less than 1 micro-second, which makes it useful for resolving lesions as small as 1.55 mm. A 1 micro-second alternating magnetic field will have 10 cycles of the field oscillating to generate the acoustic signal.

In various embodiments, the frequency-modulated alternating magnetic field may is modulated (from low to high) over a period of about 1 millisecond. The high end frequency may be at or above 10 MHz to generate peak efficiency in the thermal conversion. The low-end frequency is less critical, although beginning at or below 100 KHz may be necessary for some super-paramagnetic iron oxide nanoparticles.

A continuous wave AMF system was been developed for therapeutic evaluation of hyperthermia induced by SPION. The AMF device 600, as shown schematically in FIG. 6, has an applicator coil 602 of 5 cm long and 5 cm in diameter, with a center clearance of 4 cm in diameter allowing the head of a rat to be placed therein. The single-layer solenoid 602 consisted of 5.5 turns of ¼″ hollow copper tubing 603 around a Teflon substrate. The hollow copper tubing was terminated through Teflon tubing to a water chiller 604 that regulated the circulation of deionized water at a preset temperature. A heavy-duty capacitor bank 606 was placed in series with the AMF applicator coil 602 to create an inductor-capacitor (LC) network, and the resonance frequency of the LC network determined the tuning frequency of the driving circuit. A sinusoidal RF signal from a function generator 608 was amplified by a class B RF power amplifier 610 (from T&C Power Conversion, Rochester, N.Y.) that was capable of delivering 500 W to a 50 Ohm load within a FWHM bandwidth of 100 KHz-1 MHz.

Tapping terminals 612 were mounted to the solenoid coil 602 for adjusting the coupling efficiency between the RF power amplifier 610 and the resonance circuit. By different combinations of the capacitors in the bank 606, CW AMF with a frequency between 88.8 KHz to 1.102 MHz was obtained. Due to limited positioning of the tapping terminals 612, the coupling of the RF power to the coil 602 was not optimal across all frequencies of choices, and the field strengths measured at the center region of the coil 602 varied from 52 Oe (4.14 KA/m) to 220 Oe (17.5 KA/m) in the frequencies realized. The field strength was measured by placing a single turn pick-up coil 614 of 1.27 cm in diameter in the middle-section of the AMF coil 602 and converting the induced frequency-proportional voltage. An oscilloscope 616 was also attached. The temperature of the SPION sample was measured by an immerged fiber optical temperature sensor 618 connected to a multi-channel data monitor (FISO, Quebec, QC, Canada) through computer interface for continuous data acquisition.

A dextran based cross-linked iron oxide (magnetite) (CLIO) nanoparticle was used as the SPION sample for measurement of initial temperature rise under steady-state AMF mediation. Transmission electron microscopy was used to establish the average size of the dextran coated nanoparticles, which were found to have an elongated shape, with an average length of ˜10 nm. Light scattering (Nanotrakparticle size analysis) was used to establish the hydrodynamic size of the nanoparticles, which were found to have an average size of ˜120 nm. The SPION sample used for the benchtop testing has an iron-weight concentration of 0.8 mg/ml. The weight concentration of the SPION in the host medium was measured experimentally as 0.64% (an average obtained from duplicates), which corresponds to 0.0946% volume fraction of the SPION solids in the liquid matrix based on the mass densities of the magnetite and the carrier fluid as specified in Table 1.

TABLE 1
Material-specific parameters used for simulating the heat dissipation of SPION

| Symbol | Parameter specification | Value | Unit |
|---|---|---|---|
| cₐ | Speed of sound in tissue | 1550 | [m s⁻¹] |
| Cᵥ | Specific heat of carrier | 2080 | [J kg⁻¹ K⁻¹] |
| | fluid | | |
| H | Magnetic field strength | 7.96 | [KA m⁻¹] |
| Mₛ | Saturation magnetization | 446 | [KA m⁻¹] |
| Tₑₘₚ | Thermodynamic | 298 | [K] |
| | temperature | | |
| V_{M} | Magnetic volume | Corresponding to a diameter of 9.5 nm | [m³] |
| V_{H} | Hydrodynamic volume | Corresponding to a diameter of 120 nm | [m³] |
| η | Viscosity coefficient | 0.00235 | [N s m⁻²] |
| κ | Anisotropy energy density | 23 (the smallest value used by Ref. 10. | [J m⁻³] |
| | of Fe₃O₄ | The higher the value, the higher the heat | |
| | | dissipation) | |
| ρ | Mass density of Fe₃O₄ | 5180 | [kg m⁻³] |
| ρ | Mass density of carrier | 765 | [kg m⁻³] |
| | fluid | | |

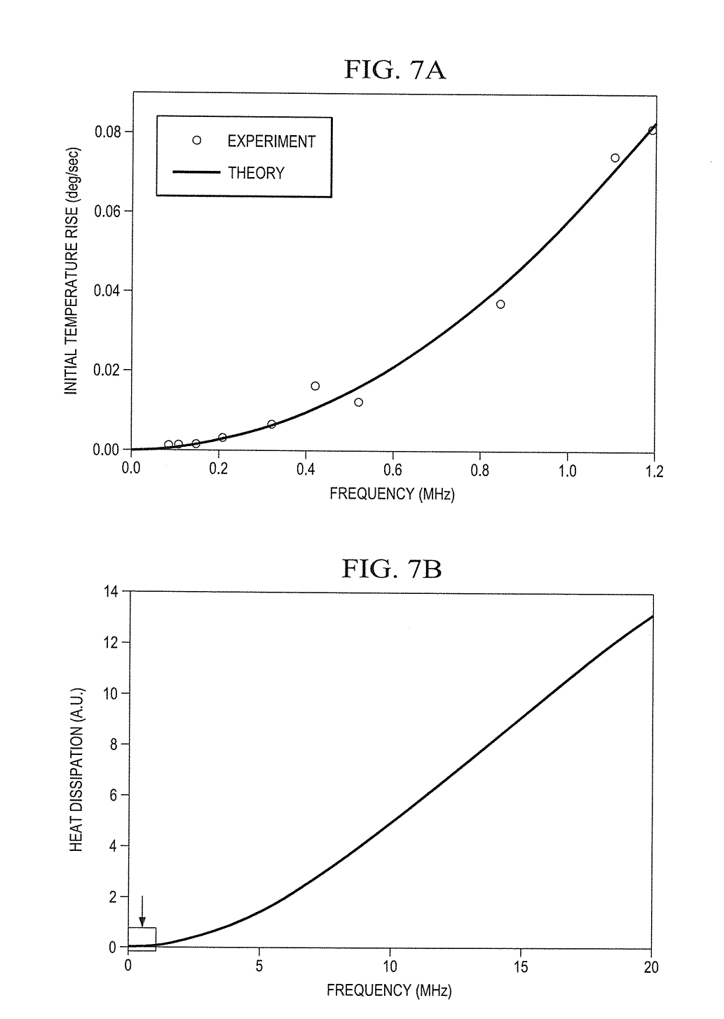

A 20-ml vial containing 5 ml of 0.8 mg/ml SPIONs was placed in the AMF coil 602 for measuring the heating of the SPION matrix under CW AMF. The initial rate of temperature rise [degree/second] was measured as the initial slope of the temperature change after the onset of AMF. Temperature was continuously monitored over the duration of AMF application that lasted between several minutes to 40 minutes depending upon the actual heating rate and the interested range of temperature measurement. Because the AMF intensities were different across the frequencies realized, the temperature rise was normalized to an AMF field intensity of 100 Oe (7.96 KA/m), based on the dependence of heat dissipation upon the square of AMF field intensity. The experimentally measured initial rates of temperature rise were than compared to the model-prediction based on Eq. (2.13) using the material and dimensional parameters detailed in Table 1. The results are shown in FIG. 7(A). The fitted model is then extrapolated to 20 MHz as shown in FIG. 7(B) (it was evaluated but not illustrated here that, as the frequency reaches 40 MHz and above, the curve levels off and eventually flattens). The heat dissipation rises monotonically with the frequency over the range shown, and a 10 times of increase of frequency from 1 MHz to 10 MHz results in approximately 84 times of increase of the heat dissipation.

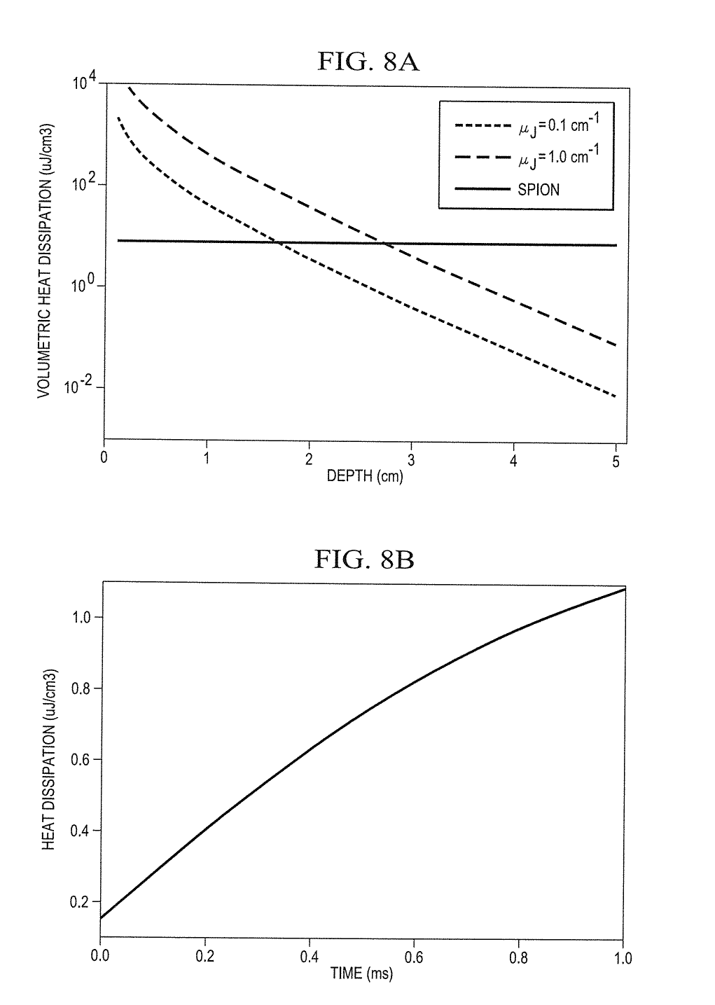

At 10 MHz AMF frequency, a 1-μs burst of AMF contains 10 complete cycles. If a 1-μs burst of 10 MHz 100 Oe AMF is applied to the same 0.8 mg/ml SPION matrix used for the experimental measurement, the volumetric heat dissipation based on Eq. (2.10.TD) is 7.7 μJ/cm³. This value corresponds to the horizontal line shown in FIG. 8(A). As the tissue attenuation to time-varying magnetic field is negligible, the AMF induced heat dissipation from SPION can be assumed depth-invariant. However, the photo-induced heat dissipation is strongly dependent upon the depth from the irradiation. In regards to the potential of thermo-acoustic wave generation, it is imperative to compare the 7.7 μJ/cm³ heat dissipation estimated for SPION under a time-domain AMF against the heat dissipation by a chromophore in tissue under a surface light illumination as strong as 100 mJ/cm². For a typical biological tissue that has a reduced scattering coefficient of μ′ₛ=10 cm⁻¹ and an absorption coefficient of μₐ=0.1 cm⁻¹, the fluence in tissue in the diffusion region due to a surface fluence Ψ₀ reduces quickly versus the depth r at a rate that is not smaller than the following one for an unbounded medium:

$${Ψ}_{r} = {Ψ}_{0} \frac{3 ( {μ}_{a} + {μ}_{s}^{′} )}{4 π} \frac{exp ( - \sqrt{3 {μ}_{a} ( {μ}_{a} + {μ}_{s}^{′} )} r )}{r} \qquad ( 3.1 )$$

The heat deposited by a chromophore of absorption coefficient μₐ^{chro} is:

Δq(r,μₐ^{chro})=μₐ^{chro}Ψ₀ (3.2)

At a surface irradiation fluence of Ψ₀=100 mJ/cm², the heat deposited by a chromophore of μₐ^{chro}=0.1 cm⁻¹ or μₐ^{chro}=1 cm⁻¹ versus the depth of the chromophore in the biological tissue of μ′ₛ=10 cm⁻¹ and μₐ=0.1 cm⁻¹, according to Eqs. (3.1) and (3.2), is shown as the dashed or dotted curve in FIG. 8(A). The case of μₐ^{chro}=0.1 cm⁻¹ corresponds to the heat dissipation in the biological tissue with no specific chromophore, whereas the case of μₐ^{chro}=1 cm⁻¹ corresponds to the heat dissipation by a chromophore that has 10 folds of absorption contrast over the background tissue. FIG. 8(A) shows that the predicted volumetric heat dissipation from the 0.8 mg/ml SPION matrix when exposed to 1 μs of 10 MHz 100 OeAMF is comparable to the heat produced at 1.75 cm depth in a typical biological tissue under 100 mJ/cm² surface irradiation, and to the heat produced at 2.75 cm depth by a chromophore of 10 times of absorption contrast over the background biological tissue under the same amount of surface illumination. In comparison, FIG. 8(B) shows the estimated heat dissipation by the 0.8 mg/ml SPION matrix when exposed to a 1-ms train of 100 Oe AMF with the frequency chirping linearly from 1 MHz to 10 MHz. The 1 ms duration is comparable to the duration of chirping the frequency of the amplitude modulation in the frequency-domain photo-acoustics. The heat dissipation of FIG. 8(B) increases monotonically from 0.15 to 1.1 μJ/cm³ over the 1 ms duration. REFERENCES
Xie J, Huang J, Li X, Sun S, Chen X, “Iron oxide nanoparticle platform for biomedical applications,” CurrMed Chem. 2009; 16(10):1278-94. Review.
Kumar C S, Mohammad F, “Magnetic nanomaterials for hyperthermia-based therapy and controlled drug delivery,” Adv Drug Deliv Rev. 2011; 63(9):789-808. Review.
Hergt R, Dutz S, Röder M, “Effects of size distribution on hysteresis losses of magnetic nanoparticles for hyperthermia,” J PhysCondens Matter.; 20(38):385214 (2008).
Frenkel J, The kinetic theory of liquids, Dover Publications, New York, 1955.
Néel L, “Influence of thermal fluctuations on the magnetization of ferromagnetic small particles,” C. R. Acad. Sci., 228:664-668 (1949).
Hergt R, Dutz S, Zeisberger M, “Validity limits of the Néel relaxation model of magnetic nanoparticles for hyperthermia,” Nanotechnology., 21(1):015706 (2010).
Balivada S, Rachakatla R S, Wang H, Samarakoon T N, Dani R K, Pyle M, Kroh F O, Walker B, Leaym X, Koper O B, Tamura M, Chikan V, Bossmann S H, Troyer D L, “AC magnetic hyperthermia of melanoma mediated by iron(0)/iron oxide core/shell magnetic nanoparticles: a mouse study,” BMC Cancer., 10:119 (2010).
Alexiou C, Jurgons R, Seliger C, Brunke O, Iro H, Odenbach S, “Delivery of superparamagnetic nanoparticles for local chemotherapy after intraarterial infusion and magnetic drug targeting,” Anticancer Res., 27(4A):2019-2022 (2007).
Amstad E, Kohlbrecher J, Müller E, Schweizer T, Textor M, Reimhult E, Triggered release from liposomes through magnetic actuation of iron oxide nanoparticle containing membranes. Nano Lett. 2011 Apr. 13; 11(4):1664-70.
Rosensweig R E, “Heating magnetic fluid with alternating magnetic fields,” J MagnMagn Mater. 2002; 252:370-374.
Silva A C, Oliveira T R, Mamani J B, Malheiros S M, Malavolta L, Pavon L F, Sibov T T, Amaro E Jr, Tannús A, Vidoto E L, Martins M J, Santos R S, Gamarra L F, “Application of hyperthermia induced by superparamagnetic iron oxide nanoparticles in glioma treatment,” Int J. Nanomedicine. 2011; 6:591-603.
Ian Baker, Qi Zeng, Weidong Li, and Charles R. Sullivan, “Heat deposition in iron oxide and iron nanoparticles for localized hyperthermia,” Appl. Phys. 2006; 99, 08H106.
Wang L V, “Tutorial on Photoacoustic Microscopy and Computed Tomography,” Selected Topics in Quantum Electronics, IEEE J 2008; 14(1):171-179.
Su R, Ermilov S A, Liopo A V, Oraevsky A A, “Three-dimensional optoacoustic imaging as a new noninvasive technique to study long-term biodistribution of optical contrast agents in small animal models,” J Biomed Opt. 2012 October; 17(10):101506.
Bowen, “Radiation-Induced Thermoacoustic Soft Tissue Imaging,” 1981 Ultrasonics Symposium, 817-822.
Kruger R A, Kopecky K K, Aisen A M, Reinecke D R, Kruger G A, Kiser W L Jr, “Thermoacoustic CT with radio waves: a medical imaging paradigm,” Radiology 1999; 211(1):275-278.
Nie L, Xing D, Zhou Q, Yang D, Guo H, “Microwave-induced thermoacoustic scanning CT for high-contrast and noninvasive breast cancer imaging,” Med Phys. 2008; 35(9):4026-4032.
Milner T E, Feldman M D, Condit C, Oh J H, “Magneto-motive ultrasound detection of magnetic nanoparticles, US Patent Application, 20090043198
Mehrmohammadi M, Oh J, Aglyamov S R, Karpiouk A B, Emelianov S Y, “Pulsed magneto-acoustic imaging,” ConfProc IEEE Eng Med Biol Soc., 2009:4771-4774 (2009).
Oldenburg A L, Gunther J R, Boppart S A, “Imaging magnetically labeled cells with magnetomotive optical coherence tomography,” Opt Lett., 30(7):747-749 (2005).
Xu Y, He B, “Magnetoacoustic tomography with magnetic induction (MAT-MI),” Phys Med Biol., 50(21):5175-5187 (2005).
Steinberg I, Ben-David M, Gannot I, “A new method for tumor detection using induced acoustic waves from tagged magnetic nanoparticles,” Nanomedicine. 2012; 8(5):569-79.
Hu G, He B, “Magnetoacoustic imaging of magnetic iron oxide nanoparticles embedded in biological tissues with microsecond magnetic stimulation,” ApplPhysLett. 2012; 100(1):13704-137043.
Nie L, Ou Z, Yang S, Xing D, “Thermoacoustic molecular tomography with magnetic nanoparticle contrast agents for targeted tumor detection,” Med Phys., 37(8):4193-4200 (2010).
Yao L, Guo G, Jiang H, “Quantitative microwave-induced thermoacoustic tomography,” Med Phys. 2010; 37(7):3752-3759.
Telenkov S, Mandelis A, Lashkari B, Forcht M, “Frequency-domain photothermoacoustics: Alternative imaging modality of biological tissues,” J. Appl. Phys. 105, 102029 (2009).
Piao D, Le K, Saunders D, Smith N, Goddard J, Figueroa D, Krasinski J S, Towner R A, “Development of a vertically and horizontally applicable multi-frequency alternating-magnetic-field device for hyperthermia of glioma in rodent model using iron oxide based nanoparticles,” Biomedical Optics and 3-D Imaging, OSA Optics and Photonics Congress, 29 Apr.-2 May 2012, Miami, Fla. Paper BSu3A.2
Towner R A, Smith N, Asano Y, He T, Doblas S, Saunders D, Silasi-Mansat R, Lupu F, Seeney C E, “Molecular magnetic resonance imaging approaches used to aid in the understanding of angiogenesis in vivo: implications for tissue engineering,” TissueEng Part A. 2010 February; 16(2):357-64.
Towner R A, Smith N, Asano Y, Doblas S, Saunders D, Silasi-Mansat R, Lupu F, “Molecular magnetic resonance imaging approaches used to aid in the understanding of the tissue regeneration marker Met in vivo: implications for tissue engineering,” Tissue Eng Part A. 2010; 16(2):365-71.
S. Purushotham and R. V. Ramanujan, “Modeling the performance of magnetic nanoparticles in multimodal cancer therapy, J. Appl. Phys. 107, 114701 (2010).
Apopa P L, Qian Y, Shao R, Guo N L, Schwegler-Berry D, Pacurari M, Porter D, Shi X, Vallyathan V, Castranova V, Flynn D C, “Iron oxide nanoparticles induce human microvascular endothelial cell permeability through reactive oxygen species production and microtubule remodeling,” Part FibreToxicol. 2009; 6:1.
Wang Y, Li C, Wang R K, “Noncontact photoacoustic imaging achieved by using a low-coherence interferometer as the acoustic detector,” Opt Lett. 2011; 36(20):3975-7.
   Thus, the present invention is well adapted to carry out the objectives and attain the ends and advantages mentioned above as well as those inherent therein. While presently preferred embodiments have been described for purposes of this disclosure, numerous changes and modifications will be apparent to those of ordinary skill in the art. Such changes and modifications are encompassed within the spirit of this invention as defined by the claims.

What is claimed is:

1. A method comprising:
providing a pulsed magnetic field;
exposing a tissue mass to the pulsed magnetic field, the tissue mass containing a region imbued with magnetic particles;
receiving an ultrasonic signal from the region imbued with magnetic particles generated by the magnetic particles under the pulsed magnetic field.

2. The method of claim 1, wherein the pulsed magnetic field is pulsed by being activated for a recurring period and deactivated for a second recurring period, the activated period comprising an amplitude modulated magnetic field.

3. The method of claim 1, wherein the amplitude modulated magnetic field has a frequency of about 10 MHz.

4. The method of claim 3, wherein the activated period is about one microsecond in duration.

5. The method of claim 2, wherein the deactivated period is about one microsecond in duration.

6. The method of claim 1, wherein the activated period has a duration including at least one complete cycle of the alternating magnetic field.

7. The method of claim 2, wherein the pulsed magnetic field is pulsed by being activated for a recurring period and deactivated for a second recurring period, the activated period comprising a frequency modulated magnetic field.

8. The method of claim 6, wherein the frequency modulated magnetic field includes a frequency that varies up to a high frequency of about 10 MHz.

9. The method of claim 1, wherein the magnetic particles comprise super-paramagnetic iron oxide nanoparticles.

10. A method comprising:
attaching magnetic particles to a target tissue region within a tissue mass;
exposing the tissue mass to a field pulse enveloped alternating magnetic field; and
reading an ultrasonic signal generated by the target tissue region containing the magnetic particles.

11. The method of claim 10, wherein attaching magnetic particles further comprises attaching magnetic nanoparticles.

12. The method of claim 11, wherein the magnetic nanoparticles comprise super-paramagnetic iron oxide nanoparticles.

13. The method of claim 10, further comprising generating a map of the target tissues based on the ultrasonic signal generated by the magnetic particles.

14. The method of claim 10, wherein the pulse enveloped alternating magnetic field comprises an amplitude modulated portion.

15. The method of claim 10, wherein the alternating magnetic field comprises a frequency modulated portion.

16. The method of claim 10, wherein a period when the magnetic field is active has a duration of at least one cycle of the alternating magnetic field.

17. A system comprising:
a magnetic field generator configured to provide a pulse enveloped alternating magnetic field to a tissue mass having a target region containing magnetic particles, the pulse enveloped alternating magnetic field; and
an ultrasonic transducer that receives an ultrasonic signal from the tissue mass representative of the target region resulting from heating and cooling of the target region from the pulse enveloped alternating magnetic field.

18. The system of claim 17, wherein the magnetic field generator provides a pulse enveloped alternating magnetic field having an amplitude modulated field.

19. The system of claim 17, wherein the magnetic field generator provides a alternating magnetic field having a frequency modulated field.
